(19) United States
(12) Patent Application Publication
(10) Pub. No.: US 2005/0113649 A1
(43) Pub. Date: May 26, 2005

(54) Method and apparatus for managing a user's health

(76) Inventor: Paul V. Bergantino, Lexington, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/901,904

(22) Filed: Jul. 28, 2004

Related U.S. Application Data
(60) Provisional application No. 60/490,697, filed on Jul. 28, 2003.
(60) Provisional application No. 60/504,393, filed on Sep. 19, 2003.

(51) Int. Cl. A61B 5/00; G06F 17/60

(52) U.S. Cl. 600/300; 128/921; 705/2; 434/127

(57) ABSTRACT

A method and apparatus for managing a user's health. In one embodiment, a diet designer for designing a dietary plan, a diet director for directing a user towards a particular meal, and a diet dissector for ascertaining the nutritional content if a user's meal choices are combined in order to provide a robust health management system that operates in real time by evaluating past diet and behavior, to recommend future diet, behavior, and health management. In some embodiments, the invention includes managing a user's health by receiving the user's diet plan, receiving user input about a meal, determining the nutritional content of the meal, and determining how that nutritional content fits into the user's target diet.

### CROSS-REFERENCE TO RELATED APPLICATIONS

This application claims the benefit of U.S. Provisional Application No. 60/490,697, entitled “Method and Apparatus for Managing a Diet,” filed Jul. 28, 2003, and U.S. Provisional Application No. 60/504,393, entitled “Method and Apparatus for Managing a User's Health,” filed Sep. 19, 2003. Both provisional applications are incorporated by reference herein.

### TECHNICAL FIELD

The present technology relates to the management of a user's health. In particular, the present technology relates to a health and diet management service and system focused on improving the health of its members.

### BACKGROUND OF THE INVENTION

Over the past several decades, the health of an average member of the population has typically declined. A variety of factors has likely contributed to this deterioration, such as lack of exercise and poor eating habits. To combat this, a variety of dieting and health techniques have come about, such as programs like WeightWatchers™. Moreover, companies have developed software tools to manage a user's diet, meals, exercise routines, etc. These software tools, however, often do not proactively interact with the user, such as by reminding the user to enter what the user has consumed recently. Instead, the user may decide to use the system for a day and then may not use the system again for several weeks. Moreover, these tools often do not adapt to a user's behavior during, for instance, a particular day. These tools instead often manage a user's diet, but they do not adjust the management of a user's diet from one meal to the next.

Thus, there remains a need for a system that can manage a user's health while proactively interacting with a user and adapting to a user's consumption, activities, feelings and behaviors in at or near real time.

### SUMMARY

The invention relates to a health management module that can manage a user's diet. Moreover, the health management module can provide guidance and/or support to a user. In one embodiment, the health management module includes a diet dissector, a diet director, a diet designer, and/or a diet management database, knowledge base and/or expert system. In one embodiment, the diet dissector ascertains the nutritional content of user's meals. The diet director can provide assistance to a user in making healthy choices that conform to a user's target diet. The diet designer can help design a dietary plan for the user. In one embodiment, health management module is implemented to mimic the interaction of one or more human diet or health experts. The health management module has the ability to communicate via a plethora of devices (phone, IM, WEB, push to talk, etc.).

In one embodiment, the health management module receives a diet plan selection from a user and subsequently creates a user record with a target diet for the user. In one embodiment, the health management module then receives user input (e.g., about a meal that the user is going to consume or has consumed) and determines the nutritional content of the meal. The health management module then enters the nutritional content of the user's meal into the user's record and compares the user's consumption with the user's target diet. Thus, the invention is capable of managing a user's diet and health in real time, and in response to user input. Therefore, the invention is capable of dynamically changing recommendations based on interaction with the user.

### BRIEF DESCRIPTION OF THE DRAWINGS

The invention is pointed out with particularity in the appended claims. The advantages of the invention may be better understood by referring to the following description taken in conjunction with the accompanying drawings in which:

FIG. 1 is block diagram of an embodiment of a system having a health management module for managing a user's diet;

FIG. 2 is a more detailed block diagram of an embodiment of the health management module of FIG. 1;

FIG. 3 is a block diagram of the health management module of FIG. 1 communicating with diet management partners; and

FIG. 4 is a flow diagram of steps performed by the health management module of FIG. 1.

### DESCRIPTION

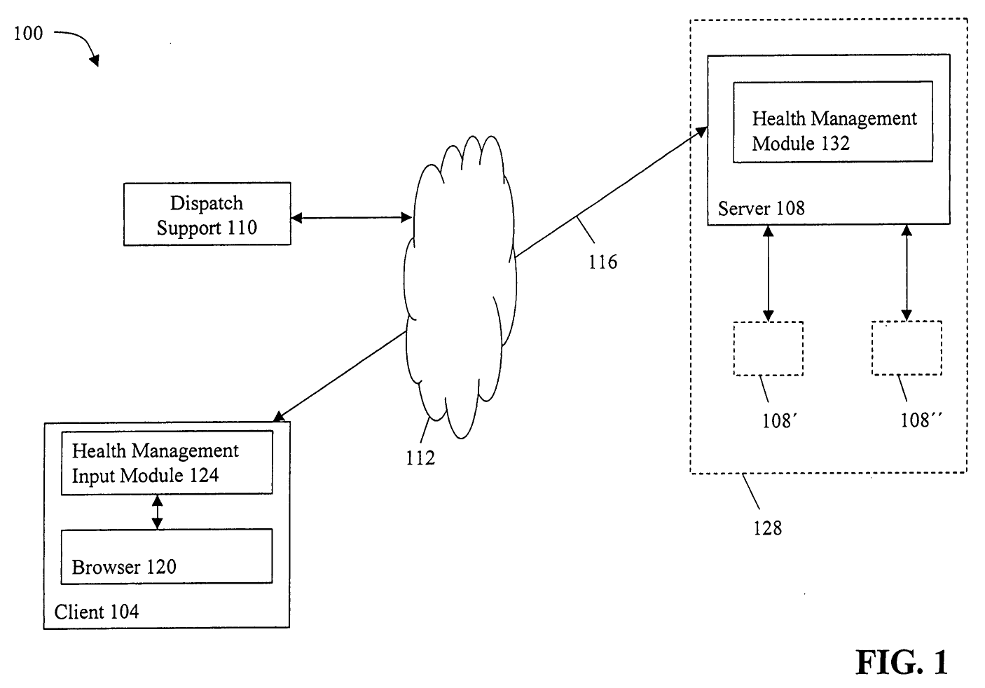

Similar to the description in U.S. Provisional Patent Application Ser. No. 60/490,697, FIG. 1 shows a block diagram of an embodiment of a health management system 100 having a client computer 104, or client, and a server 108. The client 104 is typically a personal computer that a user can use to download information from the server 108 over a network 112, such as the Internet or World Wide Web (i.e., “Web” or “web”) (e.g., wireless web). The network may be any voice network, such as a PSTN network, or any combination of networks.

The client 104 can be any personal computer, such as a smart or dumb terminal, network computer, wireless device, information appliance, workstation, minicomputer, mainframe computer, handheld device, or other computing device that has a graphical user interface. In one embodiment, the client 104 is a telephone, such as a land-line telephone or a cellular telephone. Moreover, if the client 104 is a cellular telephone, the phone may have wireless communication with the network 112 (e.g., wireless web access). In one embodiment, the phone can provide an interface by providing machine-generated prompts to the user and accepting commands and data back from the user via, e.g., a Dual-Tone Multi-Frequency (DTMF) signal, voice, phone, graphical user interface (GUI), or some combination. Further, the client 104 may also include a “walkie-talkie” or “push to talk” button that a user can assign to the server 108. Thus, if the user presses the “walkie-talkie” button, the client 104 may automatically enable the user to communicate with the server 108 (e.g., via their voice).

In one embodiment, the client 104 includes a web browser 120, such as INTERNET EXPLORER® developed by Microsoft Corporation in Redmond, Wash., to connect to the web. In one embodiment, the client 104 uses the web browser 120 to communicate with the server 108. In a further embodiment, the web browser 120 uses the existing Secure Socket Layer (SSL) support for communications to the server 108. SSL is a secure protocol developed by Netscape Communication Corporation of Mountain View, Calif., and is now a standard promulgated by the Internet Engineering Task Force (IETF). In a further embodiment, the client 104 communicates with the server 108 via an instant messaging (IM) interface using a standard IM protocol such as XMPP or SIP/SIMPLE. Voice interfaces may be provided using Voice XML. A single user may use a number of different ways to communicate with the server 108, such as by moving from desktop to home phone to fancy mobile phone with voice & data. In some embodiments, a user may simultaneously communicate with the server 108 via a plurality of modes. In this manner, the user may engage in “multi-modal” dialog with server 108 to manage the user's health. For example, in such an embodiment the user could enter what he or she consumed via his or her voice and view the consequences on a graphical display of the user's daily food and nutritional intake in real time.

The network 112 can be a local-area network (LAN), a wide area network (WAN), a network of networks such as the Internet or the Web, or a telephone network. Moreover, in one embodiment the client 104 is in communication with the server 108 over a client-server communication channel 116 that passes through the network 112. In one embodiment, the communication channel 116 is a secure communication channel. Thus, communications over channel 116 can be encrypted.

Example embodiments of the communication channel 116 include standard telephone lines, LAN or WAN links (e.g., T1, T3, 56 kb, X.25), broadband connections (ISDN, Frame Relay, ATM), and wireless connections. The connection over the communication channel 116 can be established using a variety of communication protocols (e.g., HTTP, HTTPS, TCP/IP, IPX, SPX, NetBIOS, Ethernet, RS232, messaging application programming interface (MAPI) protocol, real-time streaming protocol (RTSP), real-time streaming protocol used for user datagram protocol scheme (RTSPU), the Progressive Networks Multimedia (PNM) protocol developed by RealNetworks, Inc. of Seattle, Wash., manufacturing message specification (MMS) protocol, wireless application protocol (WAP), and direct asynchronous connections).

Additionally, the client 104 includes a health management input module 124. The health management input module 124 enables a user to transmit information about the foods the user consumes to the server 108 for analysis. The health management input module 124 can be a digital camera, a scanner, a mouse, a keyboard, a trackball, a mobile phone keypad, a user's voice, and the like. For example, if the health management input module 124 is a digital camera, the user can take a digital picture of the meal that the user is going to eat and then transmit the picture to the server 108 for real time analysis. Moreover, if the health management input module 124 is for example a scanner, the user can scan nutritional information or a product code and transmit the scanned information to the server 108.

The server 108 can be any computing device (as described above for the client 104). In a further embodiment, the server 108 is a member of a server farm 128, or server network, which is a logical group of one or more servers (e.g., servers 108, 108′, 108″ (generally 108)) that are administered as a single entity. Although the embodiment shown has three servers 108, the server farm 128 can have any number of servers 108. In other embodiments, the server farm 128 is a protected network that is inaccessible by unauthorized individuals, such as corporate Intranet, Virtual Private Network (VPN), or secure extranet.

The server 108 includes a health management module 132. The health management module 132 can, for instance, analyze food and/or drinks consumed by the user of the client 104, manage a dietary plan of the user, and/or provide dietary recommendations. In one embodiment, the health management module 132 facilitates a healthier diet, including food, nutritional intake, nutritional supplements medicine and dosage information for the user. In one embodiment, the health management module 132 is a software module. Nutritional supplements including vitamins, minerals, or other supplements may be included as part of a diet. In additional embodiments, the health management module 132 includes people to perform one or more of the functions described below.

Further, the health management module 132 can maintain dietary records for each user. Thus, as described in more detail below, a user of the client 104 uses the health management module 132 to manage his or her diet. Moreover, the health management module 132 can be used to increase the likelihood of success with an existing dietary plan, such as with Atkins Nutritional Approach™, the Zone™, Weight Watchers™, and/or Jenny Craig™. For example, the health management module 132 can track and manage the client user on one or more of these diets. Generally, the health management module translates a particular diet into a set of macro and micro-nutritional parameters, guidelines, and rules to create a diet template that is stored in a diet template database located within the health management module 132. In one embodiment, the health management module 132 may include several different databases, each for storing a particular category of data, such as a user's past food consumption, activity level or schedule, or a nutritional breakdown of particular items of food.

The health management module 132 creates and stores different templates for different diets. These templates parameterize any diet into a set of codes which can be applied to any of the future suggestions that the health management module 132 provides. In this way, the health management module 132 can advise anyone, no matter what diet they may be on or what their preferred tastes may be.

In one embodiment, the user can use the health management input module 124 to take a picture of, for instance, items that the user is about to consume or has consumed, information about the item(s), the restaurant that the user is about to enter or leave, etc. In some embodiments, the user may take a picture of items that are only similar to items that the user is about to consume. Thus, in this embodiment, the health management input module 124 is a digital or analog camera. The health management input module 124 can also be a voice recorder or have the capability to record a voice so that the user can record a description about any of the above information (e.g., items that the user is about to or has consumed).

In one embodiment, health management system 100 may include communication between client 104 and dispatch support 110 via network 112. Dispatch support 110 may in some embodiments include a call center, human coach, member or the system community, or any combination thereof. Information outputted by dispatch support 110 may be sent to client 104 health management module 132, or both. In one embodiment, once a particular piece of information, for example the nutritional content of a slice of pizza, is sent from dispatch support 110 to health management module 132, it may be stored so that in the future health management module 132 will be able to determine the nutritional content of a slice of pizza without the need to contact dispatch support 110. In another embodiment, the dispatch support 110 may contact a nutritionist in the event that the user is or may consuming an item that deviates from a specified diet plan. In another embodiment, the dispatch support 110 may notify a community member, such as the user's spouse or co-worker if for example the user has exceeded his or her daily caloric intake. Additionally, in some embodiments the user may not be aware that health management module 132 contacted dispatch support 110 and therefore it may appear that the information is originating with health management module 132 when in fact it originated from for example a community member contacted by dispatch support 110.

Furthermore, in some embodiments, the health management input module 124 can also be a cellular or land-line telephone that the user can use to call the health management module 132 for, e.g., suggestions and/or encouragement, to describe how the user feels, and to contact a nutritionist (e.g., on-line) to design a dietary plan. In other embodiments, the user can utilize the client 104 to develop a diet with the health management module 132, develop a menu, develop an activity schedule, or utilize the health management module 132 for dietary suggestions. Moreover, the health management input module 124 can also be an instant messaging client. The health management input module 124 may also change, such as transitioning from a GUI to an anthropomorphic interface to a real person. Furthermore, in some embodiments, server 108 and the client 104 can be combined into a single module enabling the system to operate as a single module or device.

In another embodiment, the health management module 132 includes an exercise management module. The exercise management module can create a target workout and use this in the creation of a target diet for a user, or vice-versa. Additionally, the exercise management module may help a user work out with workout equipment (e.g., at home or at a gym). In one embodiment, the user records the exercise completed (e.g., the screen on an exercise machine showing the total number of minutes exercised and/or the difficulty level of the exercise). For example, the user can photograph the exercise information via the user's phone and then transmit this information to the exercise management module. The exercise management module can then use this information in, for example, recommended later exercise routines and/or updating the user's data.

Thus, the health management module 132 operates in real-time, constantly adjusting dietary recommendations in response to actual consumption and/or physical activity. By tracking a user's consumption, the module 132 provides the information for a user to stay on track with their diet or get back on track. Further, the health management module 132 can alert the user when the user has met the user's goals/requirements for a predetermined time period (e.g., the day, the week, etc.). Moreover, the health management module 132 can help coach the user to prevent consuming items that may cause the user to exceed the guidelines of a particular diet plan.

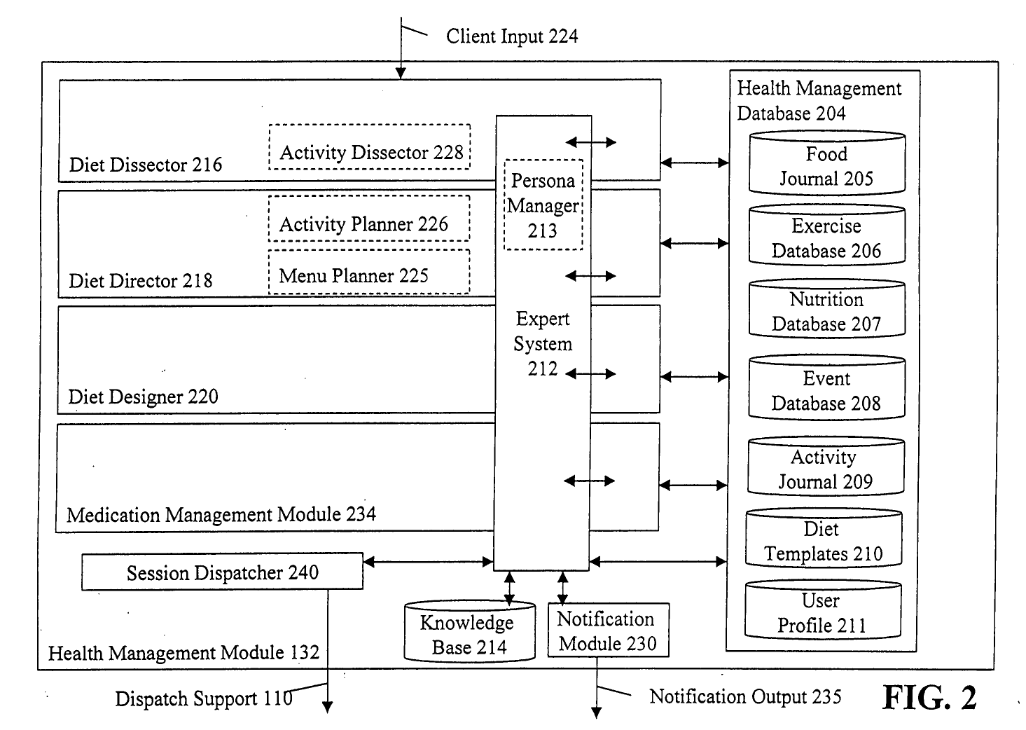

Referring to FIG. 2, the health management module 132 may in some embodiments include or be in communication with a health management database 204. Generally, health management database 204 includes the stored location of all health management data inputted to health management module 132. For example, the health management database 204 can maintain a dietary record of the foods, beverages, vitamins, minerals, amino acids, fatty acids, nutrients (e.g., basic nutrients or essential nutrients), fat, trans-fat, protein, carbohydrates, fiber, water, etc. that the user has consumed. In various embodiments, this data may be maintained in one or more of the food journal 205, exercise database 206, nutrition database 207, event database 208, activity journal 209, diet templates 210, user profile 211, or any combination thereof. Generally, health management database 204, may store any information associated with the user, such as the blood sugar level of the user who may be diabetic. This type of information may for example be stored both before and after meals. In some embodiments, the data may originate with client 104 or dispatch support 110.

The health management database 204 manages and maintains a record of information for each client user. This information may include dietary information, taste, sex, age, height, geographic location, budget, or any demographic or physiological information about the user. The health management database 204 may for example also maintain dietary records of the food, beverages, vitamins, minerals, amino acids, fatty acids, basic or essential nutrients, fat, trans-fat, protein, carbohydrates, fiber, water, medication, sodium, cholesterol or other items that the user has consumed. In some embodiments, health management database 204 may include a food journal 205. Generally, food journal 205 may include dietary information such as a record of the items that the user has consumed. In some embodiments, the health management database 204 may include one or more of an exercise database 206 and a nutrition database 207. Generally, exercise database 206 may store information regarding a dietary plan of the user, weight information, information about an exercise program, or any combination thereof. Typically, nutrition database 207 may include data regarding nutritional information or the chemical composition of food items. For example, nutrition database 207 may store the number of calories in a cheeseburger, or the grams of carbohydrates in a slice of pizza. The nutrition database 207 may also store information regarding the biodiversity of the food items. This information can be used to guide the user to food choices that deliver a diversity of healthful components and to insure that the user achieves a set of micro-dietary goals. In one embodiment, the nutrition database 207 lists the number of servings of each food group contained in one serving of the item. The definition of a food group may be defined to include any group of foods. In one embodiment, food groups supported are limited to those defined in the USDA food pyramid. In one embodiment, one or more of these food groups are further refined to offer greater granularity in measuring dietary variety in meal recommendations. For example, in one embodiment, the breads and grains food group is further divided into whole grain breads, grains and non-whole grain breads and grains. Each item in the nutrition database 207 identifies the number of servings of whole grains breast and grains and the number of servings of non-whole breads and grains it contains. Each item in the nutrition database 207 contains the number of servings of each group defined by the system. The nutrition database 207 also records the parent relationship of each food group so that a diet template may make recommendations for dietary variety, using a definition of servings (for example) as a measure at any convenient level of mixture of levels and the health management module will be able to track the users consumption at those levels and use this information to guide the users conformity as best as possible.

Health management database 204 may also include in some embodiments an event database 208. Generally, event database 208 may be used to record future or forward looking events. For example, event database 208 may record the time of the next meal, or the time of the next exercise session stored in exercise database 208. Health management database 204 may also include activity journal 209 in some embodiments. Typically, activity journal 209 may contain data regarding the users health information, habits or routines. Additionally, health management database 204 may include diet templates 210, which generally include detailed information relative to a particular diet, such as a commercially available diet program, for example. Health management database 210 may also include user profile 211. Typically, user profile 211 may include information about the user, such as his or her age, physical condition, height, or weight, for example. Generally, any one database, journal, or template may access any other database or journal to manage the user's health.

In one embodiment, health management module 132 may include an expert system 212. Generally, expert system 212 is driven by a set of rules that control the decision making and logic of health management module 132. For example, expert system 212 may respond to user input into 204 by checking the impact of the additional entries in the user's dietary guidelines, and may make a recommendation to the user concerning remaining caloric intake for the next meal. In some embodiments, expert system 212 may include a persona manager 213. Typically, persona manager 213 individualizes the interface with the user. For example, persona manager 213 may, for a particular user, decide to include or act in a manner consistent with encouragement, guilt, abuse, sarcasm or any particular personality trait in order to bring the user in compliance with the dietary requirements.

In another embodiment, expert system 212 may be in communication with a knowledge base 214. Typically knowledge base 214 may include allowing the user to ambiguously specify intake and the health management module 132 is still capable of determining user food intake. For example, if the user just enters “pasta” the health management module may have stored in the user profile that the user is a 130 pound person and thus be able to accurately determine essentially the amount of pasta consumed by the user based on, for example the users weight, or the average amount of pasta entered by the user the last 5 times the user indicated he or she ate pasta. Information gleaned from this robust system may then be stored for example in knowledge base 214. Expert system 212 may also be in communication with notification module 230 and session dispatcher 240. Generally notification module 230 is based on a rule in the expert system 212, or an event from event database 208 that will cause notification module 230 to notify user of something via notification output 235 and/or session dispatcher 240. For example, in some embodiments, notification module 230 may know the user typically consumes a snack at 4 pm. In such a case, notification module 230 may notify user via notification output 235 (to client 104) that the user should have a glass of water at 4 pm instead of a cookie. In other exemplary embodiments, expert system 212 and knowledge base 214 may jointly alert session dispatcher 240 of a pending mid-afternoon snack that is contrary to the user's health management, or diet plan. Here, session dispatcher may contact dispatch support 110 and contact a human coach who may, in one example, contact the user directly, or in another example, direct health management module 132 to instruct to user to modify his or her behavior by, in one embodiment, drinking a glass of water.

In some embodiments, expert system 212 may combine with knowledge base 214 to drive the logic of health management module 132 to determine if health management module 132 can completely answer the users needs. In some embodiments, health management system 132 may require assistance from a third party (via dispatch support 110). The third party may for example include a human operated call center, a personal human coach, or a member of the community. This assistance may be requested from the session dispatcher 240. In some embodiments, persona manager 213 enables expert system 212 to interact with or “coach” the user with a particular persona matched or selected to that user. The persona gives the system an anthropomorphic feel. In some embodiments, the expert system 212 can also dispatch certain requests or events via the session dispatcher 240 to dispatch support 110. In such a case, feedback from dispatch support 110 can be used to enhance the health management database 204, knowledge base 214, food journal 205, exercise database 206, nutrition database 207, event database 208, activity journal 209, diet templates 210, user profile 211, or any combination thereof so that in the future the health management module may be able to handle a similar event or request on its own, without having to contact dispatch support 110.

All of the above modules, journals, or elements 204-240 may communicate with each other to manage a user's diet and in various embodiments, health management module 132 may include any combination of these modules, journals, or elements 204-240.

In other embodiments, health management database 204 may include or be in communication with a diet dissector 216, a diet director 218, a diet designer 220, or any combination thereof. The diet dissector 216 generally ascertains the nutritional content of user's meals. Moreover, the diet dissector 216 transmits this information into the health management database 204. For example, the user transmits client input 224 to the health management module 132. The client input 224 can be a digital photograph of the foods that the user is about to consume. The client input 224 may also include or may be a text or audio, or scanned description of the food that the user is going to consume. The diet dissector 216 receives the client input 224 (e.g., picture and/or audio description) and analyzes the input 224 to determine information about the items that the user is going to consume. This information can include quantity (i.e., number of servings) of each food item, amount of fat, protein, carbohydrates, etc. in the items being consumed, and the like. Moreover, the diet dissector 216 can transmit the information that the dissector 216 gleans from the client input 224 to the health management database 204 (e.g., for storage or for future use). In one exemplary embodiment, the health management module 132 reflects the effect of consumption of a contemplated food item would have on a users dietary status prior to its consumption by the user. This reflection may be sent to client 104 so that the user could make an informed decision on whether or not to consume the item. In one embodiment, the diet dissector 216 communicates with expert system 212, which in some embodiments may contact knowledge base 214 to ascertain the nature of the desire to consume the item, and health management module 132 may adjust its coaching according to some estimation of the users desire. In some embodiments, health management module 132 may dispatch this information to a human coach via dispatch support 110, and the human coach may for example then contact the user directly, or rely instructions to health management module 132, and health management module 132 will in turn relay those instructions to the user. Thus, the user can determine the impact of consuming a certain item relative to a given health or diet plan without actually consuming the item. In some embodiments, these forward looking user inquiries may be given a high priority, and thus health management module 132 may expedite contact with a human coach via dispatch support.

In some embodiments, diet dissector 216 may also include an activity dissector 228. In other embodiments, activity dissector is located elsewhere in health management module 132. In other embodiments, activity dissector 228 may be located elsewhere within health management module 132. Generally, activity dissector 228 can transmit information regarding various past, present, or future user activity in real time to the appropriate database or journal. For example, if the user has just rode a bicycle for 10 miles, activity dissector 228 will transmit this information to health management database 204, or, in some embodiments, to activity journal 209 for example. In some embodiments, activity dissector 228 may track physical impact on the user, such as calories burned, by referencing activity journal 209, for example. This information may be taken into account when planning activities. This embodiment demonstrates an example of a real time update of user activity that is incorporated into the management of the users health.

Generally, the diet dissector 216 may transmit data received via client input to food journal 205, exercise database 206, nutrition database 207, event database 208, activity journal 209, diet templates 210, user profile 211, or any combination thereof. These functions can be performed in an automated fashion but any time an exception occurs, the functions can route the active session to a human to take over. In one embodiment, this routing may occur via dispatch support 110. The user may not be aware that a human is now “driving” the system.

In some embodiments, the client 104 can page or otherwise communicate with the health management module 132. Upon receiving a page or communication, the health management module 132 communicates back to client 104. Moreover, the client 104 can also specify when the client 104 wants the health management module 132 to return the client's call. The user may “page” the health management module 132 when, for example, the user needs to have input from the module 132 but is not in a location conducive to initiating a discussion (e.g., on a train). In one embodiment, the diet director 218 contacts the user if certain triggers are hit, such as if the user did not report any meals and it is 4 p.m., to get the user to provide information to the diet director 218, to take medicine, to exercise, etc.

The diet director 218 generally assists a user in making healthy choices that conform to the user's target diet. In one embodiment, this assistance is prophylactic. The diet director 218 can make recommendations based on information stored in the health management database 204 for a user and/or via communication with the diet dissector 216 (e.g., transmitting the client input 224 to the diet director 218 or communicating the analysis of the client input 224 to the diet director 218).

For example, the diet director 218 can encourage a user to forego consumption (e.g., of a particular item) if the diet director 218 indicates that the user is at or above the user's target consumption levels for that period of time (e.g., for that day, for that week, etc.) In another embodiment, the diet director 218 provides suggestions on healthy choices that fit within the user's dietary plan. These suggestions can incorporate, for example, target levels of fat, protein, carbohydrates, vitamins, minerals, lipids, and water against amounts that the user has consumed to that point before the suggestion. In particular, the diet director 218 can provide recommendations, for example, about food or drink choices, food quantities, exercise routines, etc. Thus, the diet director 218 can provide recommendations about any of the information maintained in the health management database 204.

In one embodiment, the diet director 218 monitors the user's intake throughout the day and transmits a recommendation to the user when the diet director 218 determines that the user should (or should not) consume a particular item(s). The diet director 218 transmits the recommendation to the client 104. In one embodiment, the transmission of a recommendation may be a call to the user's cellular phone, a text message to the user's cellular phone, an email to the user's handheld device, and the like. Moreover, the user can solicit a meal recommendation from the server 108 with a handheld device 104 or via email, etc.

In other embodiments, the diet director 218 provides dietary recommendations in response to user inquires. For example, a user can transmit, via the client 104, a request (i.e., client input 224) to the diet director 218 for a recommendation on a particular item. The diet director 218 can then answer the request with a recommendation. In one embodiment, the diet director 218 obtains a list of recommendations from the database 204 based on the user's target diet and/or past consumption. In another embodiment, the recommendation may be based on a user's location, budget, preferences, diet and wellness (dieting variety) goals, as well as diet and past consumption. The recommendation can be generic (e.g., six ounce chicken sandwich) or specific (e.g., Wendy's® Spicy Chicken Sandwich).

Further, the diet director 218 can also coach the user. The diet director 218, supported by the expert system 212 could have an anthropomorphic persona, managed by the persona manager 213, that communicates with the user, learns about the user, and adapts its responses based on this learning of the user. For example, a dieter tempted by a donut can transmit this information to the health management module 132 (e.g., as the client input 224). In response to the notice that the user is tempted by a particular food, the diet director 218 can coach the user through the temptation or guide the user toward healthier options. In other embodiments, the diet director 218 analyzes the information associated with the user (e.g., stored in the health management database 204) and may determine that the user can eat the donut. In this case, the diet director 218 factors the donut into future recommendations.

In one embodiment, the diet director 218 includes a menu planner 225. The menu planner 225 is accessible to the user to construct menus (e.g., daily or weekly menus) that fit the user's target diet. The menu planner 225 can factor, for instance, budget, prices, target diet, variety, and/or tastes of the user in the design of a menu. The menu planner 225 can also create a shopping and/or price list as well as provide coupons for partner markets. In some embodiments, the menu planner 225 can create a recipe and a portions guide for meals that the user selects. The menu planner 225 may also allow the user to place orders with partner markets and/or allow the user latitude in planning the menu. In another embodiment, the diet director 218 may include an activity planner 226. Generally, the activity planner plans athletic or exercise activities for the user as part of the overall health management. The activity planner 226 can factor, for example, the user's physical activity, such as the fact that the user may jog for 30 minutes a day, or may be bedridden with a cold or other illness. The activity planner 226 can also create an activity list including suggested physical activity that is then factored into the users diet. For example, the activity planner 226 may allow the user to eat a donut if the user has, or will, go on a 3 mile jog.

The diet designer 220 designs dietary plans for the client user. In one embodiment, the diet designer 220 communicates with external parties, such as diet companies, local nutritionists, book authors, etc., to help develop a dietary plan. Further, the diet designer 220 can provide on-line access to a diet designing application that enables the user to develop a target diet. In some embodiments, a proxy of the client may be authorized to develop a target diet. The health management module 132 can then manage the target diet. Moreover, the diet designer 220 can enable a partner nutritionist, as described in more detail below, to enter target diet information. The diet designer 220 can also enable the nutritionist to monitor the user, such as through a status screen and/or through queries to the health management module 132. In some embodiments, health management module 132 may enable a nutritionist to monitor the user. Moreover, the health management module 132 can enable the nutritionist to provide coaching to the client users. The health management module 132 can also enable a nutritionist to use an input terminal to monitor and/or to provide or assist in providing one or more of the functions of the health management module 132. Although each module (e.g., the diet director 218) of the health management module 132 is described above as performing particular functions, any of the modules 132 can perform any of the functions described above. Although described as an automated system, the health management module 132 can escalate operation (e.g., response, coaching, etc.) to a human if needed. In some embodiments, this personality may be masked behind a selected persona, for example via persona manager 213.

The medication management module 234 can help manage a user's drug intake. For instance, the medication management module 234 can transmit reminders to a user to take a particular medication at a particular time of day, to refill a prescription, etc. Moreover, in some embodiments, the medication management module 234 provides suggestions as to the amount of a medication to take. Further, this recommendation may generally be fixed (e.g., one pill in the morning, two pills at lunch, one pill at dinner) or may be tied to another factor. For example, a diabetic user can transmit information to the health management module 132 about what the user is about to consume and the user's blood sugar before the user consumes the meal. The medication management module 234 can use information that the health management module 132 stores to make a recommendation to the user. An example includes the medication management module 234 retrieving information about the items in the meal, such as the amount of carbohydrates and sugar in each item in the meal, and about the particular user, such as the user's weight and the user's sensitivity to insulin, to recommend an insulin dosage to the user for the meal.

Other examples include the medication management module 234 recommending a particular type of medication to a user with a particular problem, enabling the user to confirm the user's doctor's recommendation on a medication to take for a certain problem, providing a description as to the side effects of a medication, and the like. In some embodiments, medication management module 234 can ensure that dietary recommendations prepare the user properly for consumption. For example, medication management module 234 will comply with a medication whose consumption is recommended on an empty stomach, or with milk or water.

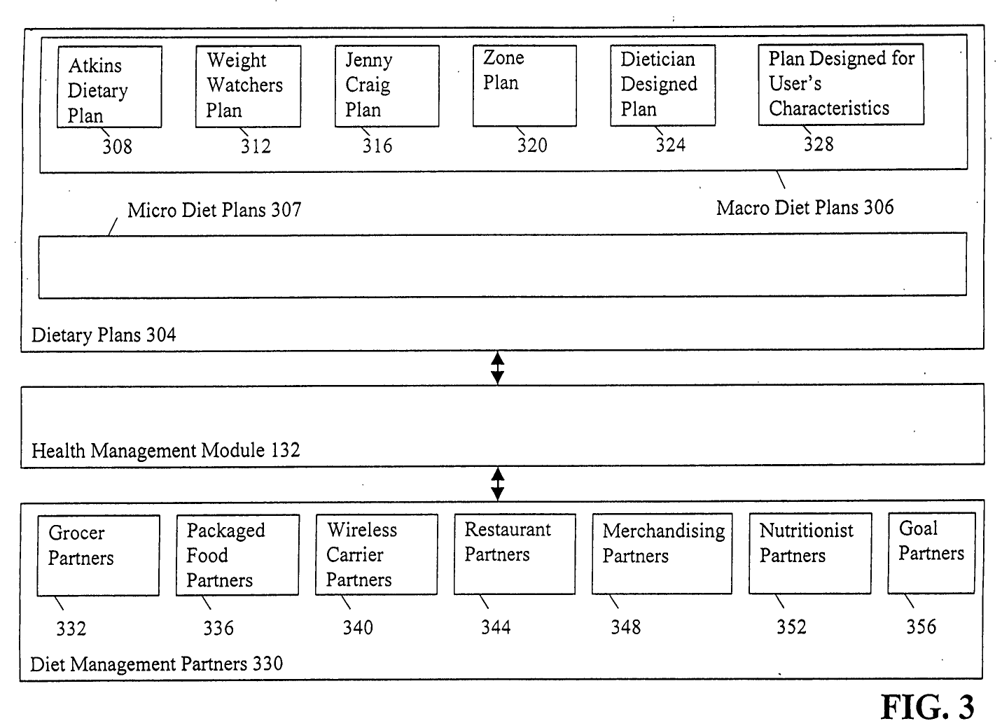

Referring to FIG. 3, in one embodiment, the health management module 132 enables a user to select (and/or receive information about) one or more diet plans 304. In one embodiment, the health management module 132 enables a user to select a macro (i.e., body mass) diet plan 306 or a micro (wellness) diet plan 307. The macro diet 306 can specify quantities of the macro nutrients (e.g., carbohydrates, proteins, and/or fat) that a user consumes on a daily basis. In one embodiment, the macro diet 306 affects the user's body mass by specifying caloric intake goals, etc. In one embodiment, the health management module 132 also provides a supplementation plan for micronutrients when the user selects the macro diet 306. The macro diet 306 can include one or more of an Atkins dietary plan 308, a Weight Watchers™ dietary plan 312, a Jenny Craig™ plan 316, a zone plan 320, a dietician designed plan 324, and/or a plan designed for user's characteristics 328. For example, a dietician may design a specific plan for a user who then wants to incorporate that plan into the user's use of the health management module 132. Thus, the health management module 132 can incorporate the dietician's plan into its recommendations to the user. Similarly, a dietary plan 304 can include a plan designed for user's particular characteristics 328, such as allergies, likings, etc. The health management module 132 can use this plan to further tailor its recommendations. Although several macro diet plans 306 are listed (e.g., the Atkins dietary plan 308), the macro diet 306 can include any dietary plan and any number of dietary plans.

Additionally, the micro diet 307 is designed to select foods that are thought to or have been found to prevent or treat a disease or diseases of particular concern to a user, or to generally improve the users health. In one embodiment, the micro diet 307 specifies the required daily intake of micronutrients, such as vitamin intake and/or mineral intake, and may also specify foods that provide the required daily amount of one or more of the various micronutrients. In some embodiments, micro diet 307 may be defined by a user-specific diet template 210. In other embodiments, generic templates may exist, for example for fighting breast cancer, and the user may select and implement one of these diet templates.

In one embodiment, the diet director 218 uses the micro diet 307 when constructing a weekly menu or making a meal recommendation. The diet director 218 can also use the micro diet 307 to preferentially list different sources of carbohydrates, fats, and proteins (e.g., favoring those foods that are rich in the compounds thought effective in preventing the disease(s) of particular concern to a user).

For example, a woman who has a history of breast cancer in her family may select a micro diet 307 that favors foods thought to help prevent breast cancer. In one embodiment, the macro diet 306 is orthogonal to the micro diet 307. A user who selects the Atkins dietary plan 308 as the user's macro diet 306 can also select a “basic nutrition plus breast cancer prevention diet” as the user's micro diet 307. Moreover, a user who selects the Zone diet 320 as the macro diet 306 can also select a “basic nutrition plus breast cancer prevention diet” as the user's micro diet 307. For both of these users, the health management module 132 makes meal recommendations that conform to both the macro diet 306 and the micro diet 307.

In one embodiment, the health management module 132 communicates with diet management partners 330 to develop and catalog food choices. The diet management partners 330 can include one or more of grocer partners 332, packaged food partners 336, wireless carrier partners 340, restaurant partners 344, merchandising partners 348, nutritionist partners 352, and goal partners 356. For example, restaurant partners 344 can transmit their menus to the health management module 132. The health management module 132 can then enter this information into the health management database 204. The health management module 132 can additionally enter nutritional content and dietary variety of each menu choice into the database 204. For example, dietary variety may include a robust serving. The health management module 132 may order recommendations based on an assessment of a users likely acceptance criteria. For example, if a user tends to select meals by price, meals may be listed by price. If the user likes chicken, chicken may be listed first. Preferences may for example, include location, cost, type of food, favorite restaurant, cuisine, other factors, or any combination thereof.

In one embodiment, the health management module 132 can recommend a particular partner for the user to travel to based on the partner's location and the user's location and or the applicability of the partner's available food choices based on the users past consumption or dietary goals. Moreover, the health management module 132 may place orders, make reservations, provide driving directions to and/or provide contact information of one or more of the diet management partners 330 (e.g., a restaurant partner 344) to/for the user. In yet other embodiments, the health management module 132 transmits a tag to the client 104. The user can then travel to a partner (e.g., a restaurant partner 344, such as a fast food partner, or a grocer partner 332) and the partner 344 can scan or read the tag. This may be done electronically, or wirelessly, for example. In one embodiment, the partner 344 transmits the scanned or read information to the health management module 132. The health management module 132 then uses the scanned information to determine food information for the items that the user purchased. In another embodiment, a tag can be used to identify a client's technology to another client 104. Thus, in one embodiment, a user of a first client 104 can scan a tag denoting the first client's technology (e.g., mobile phone) and transmit this information to a second client 104 so that the second client can then transmit and/or receive (e.g., infrared or wireless) information to or from the first client. Additionally, these communications may be invoked automatically or manually. In one embodiment, a user of a first client 104 can scan a tag that may also be used to provide a discount to the user, or to identify that the user is entitled to a specific price, or to credit a rebate to the user. In another example, a user of a first client 104 can scan a tag in order to provide a means to track the effect of the joint advertising efforts of the partner 344 and the service provider. In various embodiments, health management module 132 may provide a user with multiple tags that may be physical, electronic, permanent or temporary. For example, a temporary tag may grant the user a sale price on a meal at a restaurant for a specific date or date range. In one embodiment, tags may identify a user for promotional purposes, or a tag may deliver preferential information to user.

Moreover, the health management module 132 can communicate with packaged food partners 336 to obtain a database of nutritional content. Further, the user can use the client 104 to transmit the Universal Product Code (UPC) to the health management module 132. For example, the user can photo-scan the UPC code of a partner's product and transmit this information to the health management module 132. The health management module 132 can then add the nutritional information of the items that the user purchases to the user's consumption record. In one embodiment, information obtained from the UPC is more detailed than the information found on a dietary label. Furthermore, users can access the diet director 218 over the web to design weekly menus and/or order food through one or more grocer partners 332. Users can also access the diet director 218 to obtain a meal recommendation that conforms to their target diet with consideration given to past consumption.

Moreover, the health management module 132 can provide a data encoding system to enable partners 330 to encode dietary content on a food package. In one embodiment, the user uses the client 104 to photograph the encoded data and then transmit this data to the health management module 132. The health management module 132 decodes the encoded data to determine the nutritional data of the item. The health management module 132 then adds this data to the user's dietary record and/or to the health management database 204 and thus assists the user in selecting a meal. In some embodiments, health management module 132 can receive a scanned or electronic version of an entire menu and recommend choice to user consistent with the users dietary goals. In one embodiment, a user can scan a UPC-like code associated with the item and obtain immediate feedback on the implications of consuming that item. The feedback can be via any of the mechanisms available to the client, such as voice, IM, graphical interface, etc. In one embodiment, the information may include one or more menu items and the health management module 132 may help select an item from the menu based on any number of factors. In another embodiment information from the UPC code, or any other scanned information can be used to provide feedback to the client to determine the effect the scanned information would have on the users dietary requirements.

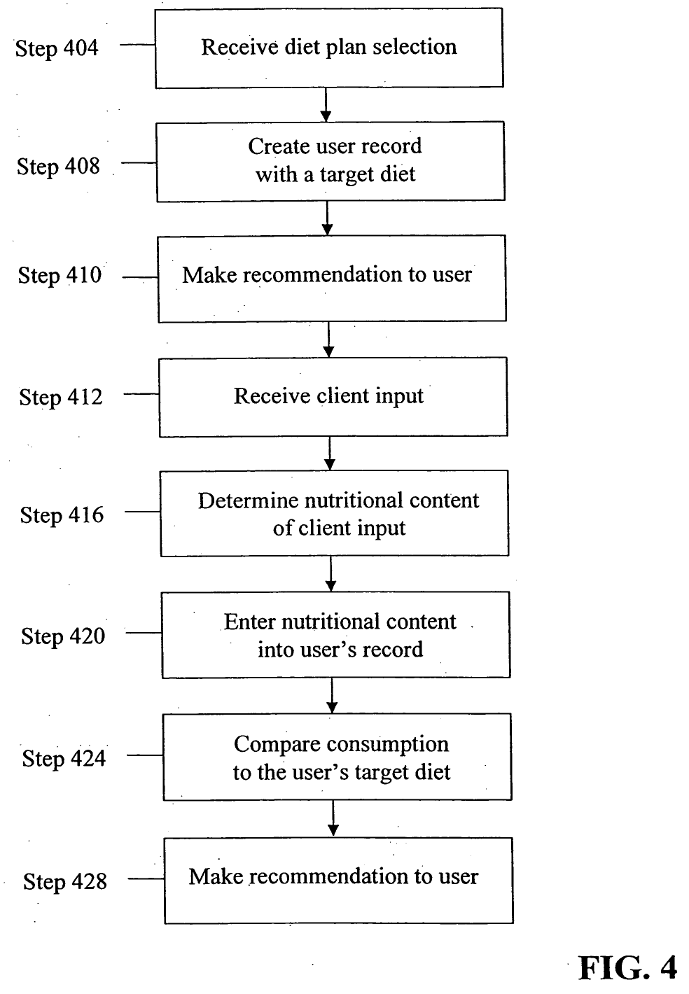

Referring to FIG. 4, the steps performed by the health management module 132 to manage a user's diet are shown. In one embodiment, the user uses the client 104 to access a web site associated with the health management module 132. The user can then read about the various diet plans 304 that the health management module 132 offers, can access any of the modules 204-240 of the health management module 132, can purchase books and/or supplements, develop a diet plan 304, etc. The user then selects a diet plan 304 that the user would like the health management module 132 to manage. The client 104 transmits the user's diet plan selection to the server 108 and the health management module 132 receives the selection (step 404).

The health management module 132 then creates a user record with a target diet for the user that is based on the diet plan chosen by the user (step 408). In one embodiment, the health management module 132 receives additional information from the user, such as personal data, budget, tastes, preferences, etc., for submission into the user's record. In one embodiment, the health management module 132 stores the user's record in the health management database 204.

The health management module 132 can then transmit one or more recommendations to the user via the client 104 (step 410). For example, the health management module 132 can inform the user, based on the user's target diet, when, where, and what to eat and/or drink. Thus, the health management module 132 can recommend to the user to drink a glass of water if the health management module 132 determines that the user needs more water to stay hydrated.

The user transmits client input 224 to the health management module 132 when the user is about to consume an item or after the user has consumed an item (e.g., some food, a drink, a vitamin, etc.). The health management module 132 receives the client input 224 (step 412) and the diet dissector 216 then analyzes each meal to determine the meal's nutritional contents (step 416). The diet dissector 216 then enters the information into the user's record (step 420). The programmatic diet dissector 216 can attempt to ascertain the nutritional content of the meal and can ask clarifying questions (i.e. by IM). The diet dissector 216 may also elevate the input to an actual human dissector for additional expertise in analyzing and recording consumption.

The diet director 218 then compares the consumption (e.g., the meal's nutritional content) to the user's target diet (step 424). For instance, the diet director 218 can compare the amount of calories that the user has consumed by eating the meal with the amount of calories that the user should have consumed from this meal. Based on this comparison, in one embodiment the health management module 132 transmits one or more recommendations to the user (step 428). For example, if a diet deficiency or diet excess (e.g., an excess daily intake with respect to the user's diet) is found, the diet director 218 can notify the user via the web and/or via the user's cell phone (e.g., using instant messaging) (i.e., via the client 104). Moreover, the diet director 218 may factor in the user's target diet, the location of the user, the user's preferences, the user's tastes, the user's budget, etc. when making a recommendation. If the diet director 218 locates an excess in the user's intake, the health management module 132 may factor this excess into later meal recommendations. Further, the diet director 218 may factor in the user's goals for dietary variety, and the decision to coach or escalate the feedback to the client 104 may also be made.

In one embodiment, the user can access the health management module 132 at any time. For example, the user can access the diet director 218 via his mobile phone for a meal recommendation. The user can also access the health management module 132 to view the user's past consumption and information associated with the user's past consumption (e.g., nutritional facts associated with the user's lunch selections).

Moreover, the user can also use the client 104 to capture a brief audio/picture synopsis of the items on hand at the user's home so that the diet director 218 can make a recommendation based on the items already in the home. The user may also provide a list of food items (e.g., once, regularly, etc.) to the health management module 124. The health management module 124 can consider the user's listed items (e.g., in view of diet variety and/or diet targets) when making a meal recommendation. If the food items around the user are tagged with a Radio Frequency Identification (RFID) tag, the user can also use a scanning or other similar device to input data through the health management input module 124, automatically identifying what food items are on hand. The health management module 132 can then use this information for meal planning and recommendations.

In another embodiment, a user who wants to eat a particular meal can ask the health management module 132 to inform the user when it is an appropriate time for the user to consume the meal. This recommendation may be based on, for example, past consumption (e.g., for the day, the past week, the past month, etc.), target diet of the user, and/or the nutritional content of the meal. For example, if a user loves to eat hamburgers, such as a WHOPPER, JR.® from BURGER KING®, in one embodiment the health management module 132 notifies the user when the user can have his or her next WHOPPER, JR.®. Thus, a user can register one or more meals of choice and/or one or more times of day that the user prefers to eat the meal of choice. The health management module 132 then notifies the user when the user can fit the meal of choice into the user's diet.

In another embodiment, invention may include a calorie clock. In some embodiments, the calorie clock may be the central status and feedback mechanism or graphical display for the user. In one embodiment, the calorie clock displays a user's caloric consumption for the day as well as for the previous week, for example. In this illustrative embodiment, the calorie clock may contain a time-of-day hand. The time-of-day hand may be an hour hand that rotates around the face of a 12-hour clock twice each day. For example, at 2:15, the hand shall be located 1/4the circumferential distance between the “2” and the “3”.

In some embodiments, the calorie clock may include a caloric-intake hand. The caloric-intake hand reflects the user's actual consumption of calories, as measured by how far into the day the calories consumed should take her. For example, if a user eats an appropriate breakfast, she should consume enough calories to cover her needs until either a morning snack or lunch time. She should not consume enough calories to take her all the way through to dinner. The goal of the calorie-intake hand is to present the user with a reflection of how far ahead or how far behind she is in terms of caloric consumption. If she is eating too much, the calorie-intake hand will show how far into the future she is eating. If she stops eating, the time-of-day hand will eventually catch up to the calorie-intake hand. She should refrain from eating until this occurs. The calorie intake hand may advance each time consumption is reported by the user.

In some embodiments, the position of the calorie-intake hand may be determined as follows: At initialization of the service, the calorie-intake hand is set to the present time. The number of minutes in a day is divided by the target caloric intake of the user. This quotient yields the user's “minutesPerCalorie” factor. Each time that the user reports consumption, the calories consumed is multiplied by the subscriber's minutesPerCalorie factor to obtain a measurement of minutes. This measurement represents an approximation of the number of minutes that it will take the user to use or “burn up” the calories. The calorie-intake hand is advanced by this number of minutes. In some embodiments, when the calorie-intake hand is ahead of the time-of-day hand the wedge of the face between them may be shaded red, and when the calorie-intake hand is behind the time-of-day hand the wedge of the face between them may be shaded green.

In another embodiment, the present technology relates to personal management including one's diet, wellness, exercise regime, hygiene, schedule, etc. In particular, the health management module 132 can display an animated character (or “animatron”) that reflects the present, past, or future state of a person (e.g., the user), animal (e.g., a pet), and/or object on the client 104 (i.e., health management input module 124). To represent the user, an image of the user may be mapped onto the animatron. The animatron may also in some embodiments assume the likeness of a stuffed animal, friend, pet, animated character, ones ideal self, or any other physical form, yet still act as a proxy for the user. The animated character can suggest actions and behaviors to maintain or modify the state of the person, animal, or object. In one embodiment, the animated character predicts, tracks, announces and/or reflects changes to the state of the person. An example of the animatron representing an object is the animatron representing an automobile. In this embodiment, the animatron can reflect scheduled maintenance and other predicted states. For example, if the auto-dealer has not recorded that the user has come in for tire service in a timely fashion, the animated automobile can reflect worn tires. The animatron for an automobile can also provide information about schedule maintenance. For instance, at each maintenance interval, the animatron can reflect or announce the likely maintenance deficiency, corrective action, and, possibly, costs of the operation and places and times to have the service performed. Therefore, the animatron may in some embodiments communicate to the user any chore or event, such as for example indicating when it is tome for car maintenance, or to paint the user's house, for example.

When the animatron is meant as a proxy for a person, the state of the person, as reflected or reported by the animatron, may be determined by, for example, reported information, calculated information, scheduled information and/or well-known information. Particular examples include information read from memory, food consumption reported by a dieting person to the health management module 132, and/or metabolic state (e.g., whether the person is burning carbohydrates, fat or protein recently consumed or burning stored fat or muscle as a result of a lesser consumption level, as calculated by the health management module 132 based on stored information about the person and reported information about consumption). In one embodiment, the animatron includes a knowledge base (such as a database of stored information). In further embodiments, the knowledge base is separate from the animatron, as with a separate database.

In one embodiment, the animatron reflects the metabolic state of a person. The animatron can demonstrate whether a person is, for example, burning stored fat and becoming “thinner” or storing excess calories in the form of fat and becoming “fatter”. In one embodiment, the animatron reflects state by shrinking or growing based on the metabolic state of the person. If the health management module determines that the person is burning fat, the animatron can shrink. The animatron may also begin singing a song, such as “I feel good”, etc. If the health management module 132 determines that the person is converting excess carbohydrates to fat, the animatron may be displayed as growing in some form. In one embodiment, the actual deformation varies based on what is shown to have the most pronounced effect on the behavior of the person for whom the animatron is a proxy.

In one embodiment, the animatron provides direction to the person. For example, if the health management module 132 determines that the user needs to drink a glass of water, the animatron “tells” the user that he should drink an amount of water. This “telling” can occur by the animatron announcing “I am thirsty”, or the animatron can be shriveled, or any combination of these. Other information that may be conveyed by the health management module 132 through the animatron is when and what to eat and drink, when to take medication, when to rest, when to exercise, when to get a haircut, when to visit the doctor, when to visit the dentist, when to attend a meeting, etc.

In one embodiment, the animatron, as directed by the health management module 132, is able to announce that the animatron is hungry and can then make recommendations on food choices. Animatrons can also announce other known scheduled requirements, such as when to take supplements. In another embodiment, the health management module 132 directs the animatron to reflect, say, the cardiovascular state of a person, perhaps with the animatron looking winded if the person has not exercised sufficiently. The health management module 132 can also use the animatron to help a user maintain a workout program, such as by keeping a log of the repetitions and amount of weight that a user is lifting in a particular exercise. The health management module 132 can, for instance, show the arms of the animatron increasing in size as the user's increases the amount of weight that the user can lift or curl.

Long term state can also be reflected in the animatron. For instance, if a person is found to have high cholesterol, his animatron can display his heart and arteries and be directed to show constricted arteries. As the person improves his or her diet, and possibly takes medication for the condition, the animatron can reflect healthier arteries.

A user's goal may be to keep his animatron healthy and happy by rectifying the causes of deficiencies reflected by the animatron. The animatron can use visual and audio means to identify the actions required to keep one healthy. The animatron can reflect the present state of the person and is able to communicate issues with the present state in a number of ways, including multimedia messages, multimedia animation including transformations, and through adjunct meters, tables, gages, etc. In one embodiment, the animatron includes a “healthy zone” and an “unhealthy zone”. Thus, if a user who was previously unhealthy is trying to become healthier by exercising more and choosing healthier foods, the animatron moves towards/into the healthy zone. Alternatively, the animatron includes visual bars (or graph) that may or may not have points associated with them. Thus, the animatron may have a diet visual bar and, as the user eats healthier foods, the animatron's diet bar increases in value. Moreover, when the animatron is in the healthy zone, the animatron may have a diet score associated with it (e.g., 100 points if in the healthy zone for a period of six months/days, 80 points for four months/days, etc.). Points may be given for, for example, macro diet 3045 goals, adherence to dietary goals, consumption timing, avoiding bingeing or skipping meals, etc. In some embodiments, the animatron illustrates statistics of the user, such as the user's weight and height. The statistics can also include goals of the user, such as the user's target weight. The visual and audio representations of the animatron can occur in any form or manner. In further embodiments, the user can select how the animatron looks (e.g., male/female, hair color, etc.) or sounds (e.g., pitch of voice, volume of voice, etc.)

The animatron can also reflect predicted state, which is state that is possible as determined by a management module if certain actions are not taken. Additionally, a user can share an animatron (or any part thereof, such as points) with another user of a health management module 132.

In one embodiment, the animatron may help a user quit smoking or lessen the amount that the user smokes. This may occur by the animatron illustrating the user's lungs and how much smoke is in them. For instance, the user can input the number of packs of cigarettes the user smokes per day and the lungs can become darker with smoke as the number increases. If the user begins to smoke less, the lungs of the animatron can become cleaner. Further, the animatron may move more towards the “healthy zone” as the user decreases the amount that the user is smoking.

The users of the system 100 have their information and state stored within the server network 128. Different collections of these users can be grouped to form communities. Some of these communities may be structured, such as those people who work together. Some communities may be more ad-hoc, such as people within a particular geography and/or age group. Some of these communities may be random.

Users may also enable the system 100 to share certain of their information with others in their community. Information shared between users may be used to form the basis for competition or ranking within the community to see who is performing “better” at their diet/health goals. Diet and health goals may be individual or set by the community. Different communities may also be able to compete with each other for health goals.

Individuals within a community may also be able to collect points based on their performance within the system 100. These points may be used to rank the user. These points may also be used in exchange for goods or merchandise.

Although the present invention has been described with reference to specific details, it is not intended that such details should be regarded as limitations upon the scope of the invention, except as and to the extent that they are included in the accompanying claims.

1. A health management module for managing a user's health comprising:
(a) a diet designer for designing a dietary plan for the user;
(b) a diet director for directing a user towards meal choices conforming to the user's dietary plan; and
(c) a diet dissector for ascertaining nutritional content of the user's meal choices, wherein the health management module adapts at least one of its designing, directing, and ascertaining in real-time.

2. The health management module of claim 1 wherein the diet dissector receives an input regarding a user's meal from a health management input module.

3. The health management module of claim 2 wherein the input further comprises at least one of a picture of at least one item in the meal, information about at least one item in the meal, an audio description describing at least one item in the meal, a request for a recommendation, and a scanned UPC code.

4. The health management module of claim 2 wherein the health management input module further comprises at least one of a camera, a recorder, and a telephone.

5. The health management module of claim 2 wherein the diet dissector analyzes the input to determine information about the meal.

6. The health management module of claim 1 wherein the diet director further comprises a menu planner that constructs menus for the user.

7. The health management module of claim 6 wherein the menu planner comprises a selector for selecting a meal based on stored user behavior.

8. The health management module of claim 1 further comprising an exercise management module that creates a target workout.

9. The health management module of claim 1 further comprising a medication management module managing medicine taken by the user.

10. The health management module of claim 1 further comprising a diet management database maintaining records of dietary information for the user.

11. The health management module of claim 10 wherein the dietary information further comprises at least one of the nutritional content of the meal choices, the dietary plan, the information about the meal choices, weight information, information about an exercise program, and health information of the user.

12. The health management module of claim 1 wherein the function of the system adapts based on the parameters of a user selected diet plan.

13. The health management module of claim 1 wherein the dietary plan further comprises at least one of a macro diet plan and a micro diet plan.

14. The health management module of claim 1 wherein at least one of the diet designer, the diet director, and the diet dissector use at least one of diet, food consumed recently, past preferences, nutritional considerations, nutritional goals, location of user, available restaurants, health of the user, wellness issues, and budget of the user.

15. The health management module of claim 1 further comprising an animated character reflecting at least one of the user's present state, past state, and future state.

16. The health management module of claim 15 wherein the animated character comprises reflecting at least one of the user's chores, events, or appointments.

17. The health management module of claim 1 further comprising the ability to make forward recommendations based on past behavior.

18. The health management module of claim 1, further comprising a knowledge base for determining the nutritional content of an incomplete user meal choice.

19. A method for managing a user's diet comprising:
(a) receiving, from a user, a diet plan selection from a plurality of diet plans;
(b) receiving user input about a meal;
(c) determining nutritional content of the meal from the user input; and
(d) determining how the nutritional content fits into the user's diet plan selection.

20. The method of claim 19 further comprising creating a record with a target diet for the user.

21. The method of claim 19 further comprising making a recommendation to the user.

22. The method of claim 19 further comprising entering the nutritional content of the meal into the user's record.

23. The method of claim 19 further comprising comparing the nutritional content of the meal to the user's target diet.

24. The method of claim 23 further comprising comparing caloric content in the meal with caloric content recommended in the user's target diet.

25. The method of claim 21 further comprising receiving a meal recommendation request from the user.

26. The method of claim 19 further comprising proactively notifying the user.

27. The method of claim 26 wherein the notifying further comprises at least one of notifying the user when the user can consume a particular meal and notifying the user to check in.

28. The method of claim 19 further comprising managing a user's drug intake.

29. The method of claim 28 wherein the managing further comprises transmitting reminders to a user to take a particular medication at a particular time of day.

30. The method of claim 19 further comprising displaying an animated character reflecting at least one of the user's present state, past state, and future state.

31. The method of claim 30 wherein the animated character comprises reflecting at least one of the user's chores, events, or appointments.

32. The method of claim 19 further comprising reflecting a metabolic state of the user.

33. The method of claim 30 wherein the animated character changes when at least one of the present state, the past state, and the future state of the user changes.

34. The method of claim 19 further comprising generating a report about at least one of the meal, the target diet, the diet plan selection, and the plurality of diet plans.

35. The method of claim 19 further comprising communicating with at least one external party to develop the target diet.
